(19) United States
(12) Patent Application Publication
(10) Pub. No.: US 2026/0001921 A1
(43) Pub. Date: Jan. 1, 2026

(54) CHIMERA AMYLASE INHIBITOR PEPTIDE FOR INSECT PEST CONTROL

(71) Applicant: COUNCIL OF SCIENTIFIC & INDUSTRIAL RESEARCH, New Delhi (IN)

(72) Inventors: Ashok Prabhakar Giri, Pune (IN); Rakesh Shamsunder Joshi, Pune (IN); Ashwini Satyajit Rane, Pune (IN)

(21) Appl. No.: 18/807,415

(22) Filed: Aug. 16, 2024

(30) Foreign Application Priority Data
Jun. 27, 2024 (IN) 202411049522

(51) Int. Cl. C07K 14/415; A01N 63/50; A01P 7/04; C12N 15/63

(52) U.S. Cl. CPC C07K 14/415; A01N 63/50; A01P 7/04; C12N 15/63

(57) ABSTRACT

Provided herein are chimeric α-amylase inhibitors with enhanced inhibition potential, genes and genetic constructs encoding the inhibitors, and vectors encompassing the genetic sequences. Also provided herein are methods of using the inhibitors and recombinant proteins namely, chimeric Amaranthaceae α-amylase inhibitor proteins, genetic sequences, genetic constructs, and vectors in the field of insect and pest control.

### CROSS-REFERENCES TO RELATED APPLICATIONS

This application claims benefit of priority under 35 U.S.C. § 119 to Indian Patent Application No. 202411049522, filed Jun. 27, 2024, which is incorporated by reference herein in its entirety.

### SEQUENCE LISTING

A computer-readable form (CRF) sequence listing having file name Sequence listing_FCON_0031_US.xml (25,291 bytes), created Oct. 6, 2023, is incorporated herein by reference. The nucleic acid sequences and amino acid sequences listed in the accompanying sequence listing are shown using standard abbreviations as defined in 37 C.F.R. § 1.822.

### TECHNICAL FIELD

The present disclosure relates to chimeric α-amylase inhibitor with improved inhibition potential for insect pests' control. The present disclosure also relates to the field of insect pests' control, using recombinant proteins namely, Amaranthaceae α-amylase inhibitor proteins, with their inhibitory potential against various amylases. More specifically, the present disclosure relates to the field of insect pests' control, in particular pests belonging to the order Coleoptera, partially or totally, by reducing the amylolytic activity of the digestive enzymes in the intestinal lumen of the pests. Other aspects of the disclosure include gene constructs containing the nucleic acid molecules that code for Amaranthaceae α-amylase inhibitors, heterologous expression methods of new molecules in the active form, and the use of these molecules for controlling insect pests.

### BACKGROUND

Insect pests are the main factor in the production losses of important species in worldwide agriculture. For example, damage caused exclusively by boll weevil belonging to the order Coleoptera can be economically devastating for cotton producers in the countries where said insect can be found, reaching millions of dollars. The traditional methods of controlling populations of insect pests are crop rotation and application of chemical and synthetic pesticides on a larger scale. However, the government and consumers have emphatically highlighted the environmental and health damage the use of chemical pesticides brings. The regulations on the production and use of said chemical agents are provided by the laws that aim at restricting the use of said agents and punishing the users. Thus, the development of alternatives to the use of chemical pesticides is considerably interesting. The biological control of insect pests of significance for agriculture, such as fungus, bacteria and other species of insects-despite being a friendly and commercially attractive alternative does not always result in efficiency desired.

Amaranthaceae α-amylase inhibitors (hereinafter referred to as AAIs) are knottin-type, small proteins of around 30 to 41 amino acids (refer Rane A S et al., International Journal of Biological Macromolecules, 163, pages 1444-1450, 2020). They selectively interact and inhibit coleopteran α-amylases. Amaranthus hypochondriacus α-amylase inhibitor (AhAI) is a well-studied inhibitor of the knottin type. AhAI comprises three antiparallel β-strands, five β-turns, and three disulfide bonds, forming hydrophobic and hydrophilic half after folding. A cystine-knot fold formed by a network of disulfide bonds in AAIs makes them highly stable against temperature and peptidase or protease. AAI architecture and loop variations near the amylase substrate-binding groove provide specificity to enzyme-inhibitor interaction. Structural analysis of Tenebrio molitor α-amylase (TMA) and AhAI complex depicted that reactive site residues of AhAI interact with active site residues of α-amylase. It involves multiple H-bonds and hydrophobic interactions resulting in the blockage of the sugar-binding pocket. Polar and charged residues are mainly involved in these interactions. AAIs are competitive inhibitors of coleopteran amylases, emphasizing the importance of AAI's conserved Arg-7 in interaction. AAIs structure can be divided into five segments (S1 to S5) between cysteine residues.

AAIs share the typical architecture of signal, pro-, and mature peptides. Amaranthaceae and Apocynaceae α-AIs, along with spider and snail venom toxins belonging to cluster-5 of what, have a propeptide in between signal and mature peptide. Propeptide is the hallmark of cystine-knot proteins. Most active α-AI proteins extracted from plants consist of only mature peptides, while signal and propeptide might be lost during the processing as a part of post-translational modifications [refer, Nguyen PQT, et al., J Biol Chem., 290, pages 31138-31150, (2015); and Chagolla-Lopez A, et. al., J Biol Chem., 269, pages 23675-23680, (1994)]. AAI propeptides are highly variable in length and sequence composition. The role of propeptide in mature peptide folding is suggested by previous studies. Price-Carter demonstrated that in ω-conotoxins, propeptide doesn't play a role in the folding. Furthermore, it was hypothesized that the conotoxin propeptide and protein disulfideisomerase catalyzes the oxidative folding of the conotoxin precursor. It has been documented that members of cluster-5 have different origins and biological functions, but they share molecular functions. Many are often ion channel or enzyme inhibitors. Propeptides' role is assumed to be versatile and differs depending on the protein family. Still, their molecular and biological function is largely undetermined.

Further, the plant-versus-pests interaction can be visualized from two aspects: from the pest's point of view, wherein the plant varies from adequate to completely inadequate as a host; and on the other hand, from the plant's point of view, wherein the smaller the number of species and abundance of pests associated therewith and the weaker the effect said pests exert on them, the greater the resistance to said pests. The alpha-amylase inhibitor (α-AIs) was a great discovery in the group of protein inhibitors. That kind of inhibitor can be isolated from different sources. The alpha-amylase inhibitor found in common beans (Phaseolus vulgaris) has a great transgenic potential aiming for the protection of plants against insects, mainly coleopteran-Bruchidae. Several alpha-amylase inhibitors had their tri-dimensional structure determined either in an isolated way or in complex with alpha-amylases, for instance, the Amaranthus hypochondriacus inhibitor and the isolated rye inhibitor. The formation of the enzyme-inhibitor complexes as in the case of αAI-1 is totally dependent upon the protein concentration, temperature and pH.

The use of genes coding for these kinds of proteins and the expression thereof in heterologous systems (transgenic plants) surmounts the difficulties caused by the use of bioinsecticides or chemical insecticides. The Domain Shuffling and site saturation mutagenesis consists of directed molecular evolution, which causes significant changes in the primary structure of the peptide molecules by means of random mutations. Purified recombinant proteins and/or peptides can be evaluated for inhibitory potential. Therefore, there is a need in the art to solve the problem regarding abusive use of chemical insecticides as well as increasing plants resistance, generating transgenic plants which are capable of expressing genes that encode for molecules having improved alpha-amylase inhibiting activity.

### SUMMARY

An object of present disclosure is to provide chimeric α-amylase inhibitor with improved inhibition potential more particularly a modified variant of Amaranthaceae α-amylase inhibitors (hereinafter referred to as AAIs) having stronger affinity towards target amylases.

Another object of the present disclosure is to provide a peptide and pro-peptide with 2 to 8 fold higher efficacy or inhibition potential.

Another object of the present disclosure is to provide a modified variant of AAI which retains specificity towards target coleopteran α-amylases.

Yet another object of the present disclosure is to provide a modified variant of AAI i.e., recombinant Amaranthus hypochondriacus α-AI with increased specific inhibition potential.

Yet another object of present disclosure is to provide formulations for pest control comprising a modified variant of AAI which retains specificity towards target coleopteran α-amylases.

Still another object of the present disclosure is to provide generating transgenic crops or plants comprising nucleic acid molecules that code for a modified variant of AAI wherein the AAI retains specificity towards target coleopteran α-amylases.

Still another object of the present disclosure is to provide methods for increasing the plants resistance, generating transgenic plants which are capable of expressing genes that encode for molecules having improved amylase inhibiting activity.

Accordingly, the present disclosure relates to a chimera inhibitor peptide against amylase comprising a polypeptide wherein at least one propeptide region and at least one mature peptide region for the production of amylase inhibiting peptides.

In an embodiment, the present disclosure discloses a chimera amylase inhibitor polypeptide having sequence selected from the group as set forth in SEQ ID NOs: 3-6.

In another embodiment, present disclosure discloses a construct comprising a promoter region operably linked to a nucleotide sequence encoding the sequences as set forth in SEQ ID NOs: 1-2 and 7-8.

In yet another embodiment, the present disclosure discloses a vector comprising the polynucleotide sequence selected from the group consisting of SEQ ID NO. 1, 2, 7 and 8.

In still another embodiment, the present disclosure provides a transformed cell containing said construct.

In another embodiment, the present disclosure discloses a biodegradable pesticidal composition characterized by an effective concentration of the isolated chimera inhibitor peptide of the present disclosure or an analogous mutant, in an agronomically acceptable carrier.

The present disclosure, in a preferred embodiment discloses a chimera inhibitor peptide against alpha-amylase comprising a polypeptide wherein at least one propeptide region and at least one mature peptide region for the production of α-amylase inhibiting peptides or α-amylase inhibitors.

The present disclosure discloses chimera inhibitor peptide against α-amylase comprising a polypeptide wherein at least one propeptide region and at least one mature peptide region for the production of Amaranthaceae α-amylase inhibitors.

The present disclosure covers isolated nucleic acid molecules comprising: a) sequences substantially similar to any of the sequences selected from the group identified as SEQ ID Nos: 1-2; b) complements to the sequences described in SEQ ID NOs: 1-2; c) reverse complements to the sequences described in SEQ ID Nos: 1-2; d) reverse sequences of the sequences described in SEQ ID Nos: 1-2.

The present disclosure covers isolated nucleic acid molecules comprising: a) sequences substantially similar to any of the sequences selected from the group identified as SEQ ID Nos: 7-8; b) complements to the sequences described in SEQ ID NOs: 7-8; c) reverse complements to the sequences described in SEQ ID Nos: 7-8; d) reverse sequences of the sequences described in SEQ ID Nos: 7-8.

In an embodiment, the present disclosure relates to a transformed cell characterized by containing a gene construct or a binary vector containing the nucleic acid molecules of the present disclosure; or a polypeptide of the present disclosure.

The chimera inhibitor peptide amino acid sequence is identified as SEQ ID NOs: 3-4. In an embodiment, present disclosure provides chimera inhibitor peptide wherein at least one pro-peptide region is operationally linked to at least one mature peptide region for production of α-amylase inhibiting peptides or α-amylase inhibitors with higher efficacy or inhibition potential.

In an embodiment, the present disclosure provides an inhibitor peptide with a mutation in the at least one mature peptide region wherein said mutation selected from the group consisting of N6L, N6M, K11I, N30L, N30M, N30V in SEQ ID NOs: 5-6.

In an embodiment, the present disclosure provides an inhibitor peptide with a mutation in the at least one mature peptide region wherein said mutation selected from the group consisting of N6L, N6M, K11I, N30L, N30M, N30V in SEQ ID NOs: 7-8.

The present disclosure also provides DNA encoding the above chimera inhibitor peptide.

Furthermore, the present disclosure provides a vector, particularly an expression vector, comprising the above DNA.

The present disclosure also provides a host which has been transformed by the above expression vector.

### BRIEF DESCRIPTION OF THE ACCOMPANYING DRAWINGS

FIGS. 1A-1E illustrate the precursor AAIs. FIG. 1A depicts the sequence alignment and comparison of AAIs from Amaranthus hypochondriacus, Alternanthera sessilis, Beta vulgaris, and Chenopodium quinoa. FIG. 1B depicts an SDS PAGE gel image. FIG. 1C depicts the Western blot analysis of purified recombinant precursor α-AIs from Chenopodium quinoa, Alternanthera sessilis and Amaranthus hypochondriacus. The accurate molecular weight of AsAI_P is depicted in FIG. 1D and CqAI_P is depicted in FIG. 1E.

FIGS. 2A-2E depicts inhibition of different amylases rTcAmy, rCcAmy, porcine pancreatic α-amylase (PPA), Human salivary α-amylase (HSA), Human pancreatic α-amylase (HPA), Bacillus licheniformis α-amylase (BlAmy), Aspergillus oryzae α-amylase (AoAmy), diastase, Hordium vulgarae α-amylase (HvAmy), Helicoverpa armigera α-amylase (HaAmy) and diastase with recombinant precursor α-AIs (FIG. 2A) and graphs of [S] vs. Vo for CqAI_P (FIG. 2B), AhAI2_P (FIG. 2C), and AsAI_P (FIG. 2D) showing Ki of inhibition. FIG. 2E depicts the percent inhibition of rCcAmy and crude CcAmy of an adult by mature and precursor α-AIs.

FIG. 3A illustrates a graph showing the accurate molecular weight of CqBvAI with SDS-PAGE gel and Western blot image. FIG. 3B graphically depicts inhibition of coleopteran (rTcAmy, rCcAmy, crude CcAmy) and mammalian (human salivary, porcine pancreatic) α-amylases by CqBvAI.

FIGS. 4A-4C illustrate the SDS-PAGE gel of expressed AhAI and its mutants (FIG. 4A), a comparison of the inhibitory potential of rAhAI and its mutants against rTcAmy (FIG. 4B) and the percent inhibition of Coleopteran (TcAmy and CcAmy) and mammalian (HPA and HSA) α-amylases with AhAI and its mutants (FIG. 4C).

### DETAILED DESCRIPTION

The term “genetically modified”, as used herein refers to any change in the endogenous genome of wild type cell or to addition of non-endogenous genetic code to wild type cell, e.g., introduction of heterologous gene. More specifically, such changes are made by hand of man through the use of recombinant DNA technology or mutagenesis. The changes can involve protein coding sequences or non-protein coding sequences such as regulatory sequences as promoters or enhancers.

The term “nucleic acid” is intended to include nucleic acid molecules, e.g., polynucleotides which include an open reading frame(s)(hereinafter referred to as ORF or ORFs) encoding a polypeptide, and can further include non-coding regulatory sequences, and introns. In addition, the terms are intended to include one or more genes that map to a functional locus. In addition, the terms are intended to include a specific gene for a selected purpose. The gene can be endogenous to the host cell or can be recombinantly introduced into the host cell.

The phrase “operably linked” means that nucleotide sequence of nucleic acid molecule or gene of interest is linked to regulatory sequence(s) in a manner which allows for expression (e.g., enhanced, increased, constitutive, basal, attenuated, decreased or repressed expression) of the nucleotide sequence and expression of a gene product encoded by the nucleotide sequence (e.g., when recombinant nucleic acid molecule is included in recombinant vector, as defined herein, and is introduced into microorganism).

The term “recombinant nucleic acid molecule” includes a nucleic acid molecule (e.g., a DNA molecule) that has been altered, modified or engineered such that it differs in nucleotide sequence from the native or natural nucleic acid molecule from which the recombinant nucleic acid molecule has been derived (e.g., by addition, deletion or substitution of one or more nucleotides). Advantageously, a recombinant nucleic acid molecule (e.g., a recombinant DNA molecule) includes an isolated nucleic acid molecule or gene of the present disclosure.

The term “isolated nucleic acid molecule” is employed to relate to nucleic acids of present disclosure. Said term, when related to DNA of present disclosure, is relative to DNA molecule originated from recombination of two DNA molecules (encoding propeptide and mature peptide). For instance, “isolated nucleic acid molecules” can be inserted into vectors, such as viral vector or plasmid, or integrated with gene DNA of prokaryote or eukaryote. An “isolated nucleic acid molecule” may also comprise cDNA molecules. An isolated nucleic acid molecule inserted into a vector is herein referred to as a recombinant nucleic acid molecule. The term “isolated nucleic acid molecule” may also refer to RNA molecules transcribed from DNA molecules as described above.

The term “mutated nucleotide sequence” or “mutation” or “mutated polypeptide/peptide sequence” relates to the nucleotide sequence or amino acid which has been mutated or changed to contain one or more nucleotides amino acid residues (for example: base pairs), and is not present in the wild-type or in the non-mutated sequence. Said mutagenesis or change consists of one or more additions, deletions, or substitutions or reallocation of nucleic acid or amino acid residues.

The terms “host cell” and “recombinant host cell” are intended to include a cell suitable for genetic manipulation, e.g., which can incorporate heterologous polynucleotide sequences, e.g., which can be transfected. The cell can be a prokaryotic or a eukaryotic cell. The term is intended to include progeny of the cell originally transfected. In particular embodiments, a cell is a prokaryotic cell, e.g., a bacterial cell. Particularly, the term recombinant host cell is intended to include a cell that has already been selected or engineered to have certain desirable properties and suitable for further modification using the compositions and methods of the disclosure. The terms “pro-protein”, “pro-polypeptide” and “pro-protease”, herein refer to protein comprising mature form operationally linked to pro-polypeptide. “Pro-polypeptide” is encoded by “pro-polynucleotide”.

A nucleic acid or polypeptide is “operationally linked” when it is placed into a functional relationship with another nucleic acid or polypeptide sequence, respectively. For example, promoter or enhancer is operably linked to coding sequence if it affects the transcription of sequence; ribosome binding site is operably linked to coding sequence if it is positioned so as to facilitate translation; or modified or heterologous pro region is operably linked to a mature region of a protease if it enables the processing of the full-length protease to produce mature active form of the enzyme. Generally, “operably linked” means that the DNA or polypeptide sequences being linked are contiguous. In some instances, “operably linked” encompasses indirect linking.

The term “about” is used herein to mean approximately, in the region of, roughly, or around. When the term “about” is used in conjunction with numerical value/range, it modifies that value/range by extending boundaries above and below numerical value(s) set forth. In general, the term “about” is used herein to modify numerical value(s) above and below the stated value(s) by variance of 20%.

As used herein, the phrase “increased activity” refers to any genetic modification resulting in increased levels of enzyme in the host cell. As known to one of ordinary skill in the art, enzyme activity may be increased by increasing level of transcription, either by modifying promoter function or by increasing gene copy number, increasing translational efficiency of enzyme messenger RNA, e.g., by modifying ribosomal binding, or by increasing stability of enzyme protein, which because half-life of the protein is increased, will lead to more enzyme molecules in the cell. All of these represent non-limiting examples of increasing activity of enzymes.

Genetic knockdown includes techniques by which expression of one or more of an organism's genes is reduced. In genetic knockout, the gene is made inoperative. The present disclosure discloses chimera inhibitor peptide against amylase comprising polypeptide wherein at least one propeptide region and at least one mature peptide region for production of amylase inhibiting peptides. The present disclosure, in preferred embodiment discloses chimera inhibitor peptide against alpha-amylase comprising polypeptide wherein at least one propeptide region and at least one mature peptide region for production of α-amylase inhibiting peptides or α-amylase inhibitors.

The present disclosure discloses a chimera inhibitor peptide against α-amylase comprising a polypeptide wherein at least one propeptide region and at least one mature peptide region for the production of Amaranthaceae α-amylase inhibitors.

In an embodiment, the present disclosure discloses a chimera amylase inhibitor peptide having sequence selected from the group as set forth in SEQ ID NOs: 3-6.

In another embodiment, said amylase inhibitors are selected from Amaranthaceae α-amylase inhibitors.

In still another embodiment, said inhibitor peptide is with a mutation in one or more mature peptide region wherein said mutation selected from the group consisting of N6L, N6M, K11I, N30L, N30M, N30V in SEQ ID NOs: 5-6.

In yet another embodiment, the corresponding polynucleotide sequence is selected from the group consisting of SEQ ID NO. 1, 2, 7 and 8.

In still another embodiment, said polynucleotide sequence is cloned in vector selected from the group comprising of pET28a (Novagen, Madison, USA)

In yet another embodiment, the present disclosure discloses a vector comprising the polynucleotide sequence selected from the group consisting of SEQ ID NO. 1, 2, 7 and 8.

In another embodiment, present disclosure discloses a construct comprising a promoter region operably linked to a nucleotide sequence encoding the sequences as set forth in SEQ ID NOs: 1-2 and 7-8.

In yet another embodiment, said construct comprises a promoter optionally linked to a leader sequence and operationally linked to an encoding sequence substantially similar to one of the sequences as set forth in SEQ ID NOs: 1-2 and 7-8; a termination signal; a replication origin; a selection marker; and a cloning site.

In still another embodiment, the present disclosure provides a transformed cell containing said construct.

In another embodiment, the present disclosure discloses a biodegradable pesticidal composition characterized by an effective concentration of the isolated chimera inhibitor peptide of the present disclosure or an analogous mutant, in an agronomically acceptable carrier.

More specifically, in an embodiment, the present disclosure relates to isolated nucleic acid molecules comprising: a) sequences substantially similar to any of the sequences selected from the group identified as SEQ ID Nos: 1-2; b) complements to the sequences described in SEQ ID NOs: 1-2; c) reverse complements to the sequences described in SEQ ID Nos: 1-2; d) reverse sequences of the sequences described in SEQ ID Nos: 1-2. In an embodiment, the present disclosure relates to isolated nucleic acid molecules comprising: a) sequences substantially similar to any of the sequences selected from the group identified as SEQ ID Nos: 7-8; b) complements to the sequences described in SEQ ID NOs: 7-8; c) reverse complements to the sequences described in SEQ ID Nos: 7-8; d) reverse sequences of the sequences described in SEQ ID Nos: 7-8.

In an embodiment, the present disclosure relates to a transformed cell characterized by containing a gene construct or a binary vector containing the nucleic acid molecules of the present disclosure; or a polypeptide of the present disclosure.

In an embodiment, present disclosure provides chimera inhibitor peptide wherein at least one propeptide region is operationally linked to at least one mature peptide region for production of α-amylase inhibiting peptides or α-amylase inhibitors with higher efficacy or inhibition potential.

In an embodiment, the present disclosure provides an inhibitor peptide with a mutation in the at least one mature peptide region wherein said mutation selected from the group consisting of N6L, N6M, K11I, N30L, N30M, N30V in SEQ ID NOs: 5-6.

In an embodiment, the present disclosure provides an inhibitor peptide with a mutation in the at least one mature peptide region wherein said mutation selected from the group consisting of N6L, N6M, K11I, N30L, N30M, N30V in SEQ ID NOs: 7-8.

The present disclosure also provides DNA encoding the above chimera inhibitor peptide.

Furthermore, the present disclosure provides a vector, particularly an expression vector, comprising the above DNA.

Present disclosure also provides a host which has been transformed by the above expression vector.

Furthermore, the present disclosure provides method of producing a chimera inhibitor peptide by culturing the above host, and harvesting said chimera inhibitor peptide from said culture. In another aspect of the present disclosure, there is provided an expression vector comprising DNA fragment capable of being transcribed into a molecule that is translated by cellular machinery to produce a polypeptide.

In accordance with technology illustrated by the present disclosure, the non-specific reactions between amylase and its inhibitor, which always provide problems in the methods employing protein-inhibitor reactions, can be reduced, the reactivity of specific reactions between amylase and inhibitor can be enhanced, and enhanced function of a peptide inhibitor can be applied for formulating compositions for pest control by inhibiting coleopteran α-amylases or developing pest resistant transgenic crops or plants that can inhibit coleopteran α-amylases.

The present disclosure relates also to a biodegradable pesticidal composition characterized by an effective concentration of the isolated chimera inhibitor peptide of the present disclosure or an analogous mutant, in an agronomically acceptable carrier.

The disclosure relates to method for controlling a pest characterized by comprising steps: a) detecting the occurrence of the pest in an environment; b) promoting the contact of the pest with an isolated chimera inhibitor peptide or with a composition of the disclosure, wherein said chimera inhibitor peptide consists of the sequences selected from a group of amino acid sequences described in SEQ ID NOs: 3-6.

As employed herein, “variant” or “substantially similar” or even “analogous peptide” or “analogous mutant” comprises amino acid or nucleotide sequences different from specifically identified sequences, wherein one or more nucleotides or amino acid residues is deleted, substituted or added and may have its biological activity altered, aided, increased or reduced when compared to the native or non-mutated parental protein. The variants can be naturally-occurring allelic variants or non-naturally occurring variants. The variant or substantially similar sequences are relative to fragments of nucleic acids or peptides which can be characterized by the identity percentage of their sequences if nucleotides or amino acids with the nucleotide sequences (SEQ ID NOs: 1-2 and 7-8) or amino acid sequences (SEQ ID NOs: 3-6) described herein, as determined by common algorithms employed in the art. The preferred fragments of nucleic acids or peptides are those which nucleotide sequences or amino acid sequences have at least about 40 or 45% sequence identity, preferably about 50% or 55% sequence identity, more preferably about 60% or 65% sequence identity, more preferably about 70% or 75% sequence identity, more preferably about 80% or 85% sequence identity, even more preferably about 90%, 91%, 92%, 93%, 94%, 95%, 96%, 97%, 98% or 99% sequence identity when compared to the reference sequence. The percentage identity is determined by the alignment of two sequences to be compared, determining the number of identical residues in the aligned portion, dividing the number obtained by the total number of residues in the researched sequence and multiplying the result by 100. Said alignment can be performed through public domain tools, such as BLASTN and BLASTP, available on NCBI website (http://www.ncbi.nlm.nih.gov). The sequence alignment and the identity percentage calculation of the present disclosure have been performed as per the sequences filed with the GenBank. The term “analogous synthetic” as cited herein relates to the modification of the nucleotide sequences of the variants selected. Modifications to nucleotide base substitutions appropriating to the preferred base codon, inherent in the plant utilized in genetic transformation. The new sequence is obtained by means of chemical synthesis, and nucleotide substitutions do not represent alteration in translated amino acid sequence.

As employed herein, the terms “coding”, “encoding” or “encoded” when employed in the context of a specific nucleotide sequence means that said sequence has an information, which will be biologically translated from the nucleotide sequence into a specific protein sequence. The information with which a protein is encoded is specified by the use of codons. Said codons are explored by each living organism in different ways and different portions of nucleotide sequences may be biologically translated into identical codons. The term “gene” corresponds to a specific nucleotide sequence located at a particular region of the chromosome and is responsible for encoding a specific final product. The gene also has in its primary structure all information for processes of biological transcription and translation, such as transcription promoting and regulating regions. In the present case, gene comprises the coding nucleotide sequences corresponding to the inhibitors (αAIs) from Amaranthus hypochondriacus, Alternanthera sessilis, Beta vulgaris, and Chenopodium quinoa and to analogous gene thereof.

The terms “polypeptide”, “peptide” and “protein” are used in an interrelated way to refer to an amino acid residue polymer. The terms apply to amino acid polymers wherein one or more amino acid residue is an artificial chemical analog of a corresponding naturally-occurring amino acid, as well as to naturally-occurring amino acid polymers. The polypeptides of the disclosure can be produced either by a nucleic acid herein described or by using standard molecular Biology techniques. For instance, a truncated protein of the disclosure can be produced by the expression of a recombinant nucleic acid of the disclosure in an appropriate host cell, or alternatively by the combination of procedures, such as digestion utilizing protease and purification.

In the present disclosure the term “chimeric peptide” or “chimera inhibitor peptide” relates to the recombinant molecules resulting from the application of the gene's DNA Shuffling or domain shuffling technique (encoding alpha-amylase inhibitors). Said molecules can be expressed in, but not limited to, phages, bacteria, yeasts and plants. Preferably, the recombinant protein or chimera inhibitor peptide of the present disclosure is characterized by inhibiting alpha-amylase.

The term “substantially pure” relates to preparations comprising at least 50-60 weight % of the component of interest (for instance, nucleic acid, oligonucleotide, polypeptide, protein etc). Preferably, the preparation comprises at least 75 weight %, and more preferably 90-99 weight % of the component of interest. Purity is measured by means of methods appropriate for the component of interest (for instance, mass spectrometry and the like).

“Isolated gene” is also utilized in the present disclosure. Said term relates to the nucleotide sequence existing in a certain genome, specifically of Amaranthus hypochondriacus, Alternanthera sessilis, Beta vulgaris, and Chenopodium quinoa which codes for a protein.

The term “isolated protein” or “isolated peptide” or “isolated and purified protein” or “isolated and purified peptide” is sometimes employed in the present disclosure. Said term relates to a protein produced by the expression of an isolated nucleic acid molecule of the present disclosure. Alternatively, said term may relate to a protein that has been sufficiently separated from other proteins which it could be naturally associated with, such as it is in its “substantially pure” form. The term “isolated” does not exclude synthetic or artificial mixtures with other compounds or materials, or the presence of impurities which do not interfere with the fundamental activity of the protein, and that may be present, for instance, in an incomplete purification, addition of stabilizers, or combined within, for example, in an agriculturally acceptable composition.

The term “biological activity” is relative to a function or a group of functions performed by a molecule in a biological context (i.e., in an in vitro organism or its substitute or any other similar model). The biological activity of the proteins which are digestive enzyme inhibitors is characterized by the post-translational proteolytic processing and by the physicochemical properties such as, e.g., the presence of residues that form the active site, causing affinity (bonding or accommodation with specific enzymes). Said affinity with molecules can be produced by the mere chemical interaction between both of them.

As employed herein, term “impacting on insect pests” relates to effect of changing diet, growth, and/or behaviour of insects in any stage of development, including, but not limited to: killing insects; delaying their growth; ceasing their reproductive capacity; antifeeding activity; and so on.

“Inhibitory activity” and “insecticidal or pesticidal activity” are used synonymously to refer to activity of organism or substance (e.g.: protein) which can be measured by, but not limited to, mortality of pest, weight loss of pests, pest repellents and other behaviour and physical changes of pests after feeding and exposition for appropriate period of time. Thus, the impact of the pesticidal activity must have at least one measurable pest fitness parameter. For instance, “inhibitory and/or pesticidal and/or insecticidal peptides” are proteins which start the pesticidal activity themselves or together with other proteins. Alpha-amylase inhibitors are pesticidal proteins.

The term “improvement of the inhibitory activity” or “improvement of the pesticidal activity” characterizes a polypeptide or an alpha-amylase inhibitor of the disclosure which present the improved pesticidal activity against organism of order coleoptera in relation to the original inhibitors which are not effective against same. In order to measure the improvement of the inhibitory or insecticidal activity, one should require a demonstration of the presence or increase of the alpha-amylase inhibitory activity of at least 10% against the target insect, and more preferably 20%, 25%, 30%, 35%, 40%, 45%, 50%, 60%, 70%, 100%, 200% or a further increase of inhibition of the alpha-amylase activity regarding the inhibitory activity of other existing alpha-amylase inhibitors which are active against same insect.

The terms “inhibitor” or alpha-amylase inhibitor, or digestive enzyme inhibitor are related to a polypeptide, which presents enzymatic degradation activity with anti-nutritional and insecticidal effect. It is known from the prior art that the naturally occurring alpha-amylase inhibitors are synthesized by plants, and specifically by Amaranthus hypochondriacus, Alternanthera sessilis, Beta vulgaris, and Chenopodium quinoa.

The term “recombinantly engineered” or “engineered” relates to use of recombinant DNA technology to generate (to engineer) change in structure of the protein based on the understanding of the action mechanism thereof, and the amino acids may be introduced, deleted or substituted.

The term “DNA shuffling” is used to describe a method used in in vitro directed molecular evolution to generate variants of a sole gene sequence, or two or more homologous gene sequences through the recombination of fragments randomly generated with recovery of modified sequences and with consequent modification of amino acid residues in the encoding protein by the mutant analogous. The term “Domain Shuffling” is used to describe a method used in in vitro for shuffling or interchanging gene segments between different genes coding for functional domains and duplication, insertion, and deletion of genes that proteins principally acquire or lose domains and by which new combinations are generated.

In present disclosure, precursor peptide/protein covers the combination of pro-peptide and mature peptide.

A person skilled in the art is aware of the advances in the molecular Biology field such as a site-specific or a random mutagenesis, methodology of the polymerase chain reaction (PCR), and techniques of the protein engineering providing an extensive collection of tools and viable protocols for the use in order to change or to engineer both amino acid sequences and gene sequences disguised as proteins of agricultural interest. Therefore, the pesticidal proteins of the disclosure can be changed in many ways, including amino acid substitution, deletions, truncations, and insertions. Methods for said manipulations are generally known from the prior art. For example, the variant amino acid sequence of the pesticidal protein of present disclosure can be prepared by the introduction of mutations inside a synthetic nucleic acid (for example: DNA molecule). Methods for mutagenesis and amendments in nucleic acid are well described in the prior art. One can understand that the polypeptides of the disclosure can be produced for both the expression of a nucleic acid herein described, and by the use of standard molecular Biology techniques. One can describe that a method for controlling a pest can be characterized by the following steps: a) Detecting the occurrence of the pest in an environment; b) Promoting the contact of the pest with a pesticidal protein isolated or produced as a composition of the present disclosure.

SEQUENCES

SEQ ID NO 1:
Nucleotide sequence of CqBvAI gene:
>CqBvAI(bp)
ATCCGGGGAAAAGTCCCGTTGTTGCTCGCCCTGCGACAACGAGGAATCCCGGTG
ACACAATCGGAAGAAGCGAAAAAGATTGAGCAAGAGCTTGCAAAAATCATGCT
AGAAGCTGTTGCTGAATCTGAGTACGACAACAACAACAATAACAACAGCGCTCT
ACGTTGTATTCCACAATATAACAGGTGTGGTCCTGCAATGGATGGAGTTCCTTGT
TGTGCTCCATACACATGCACCTCAGACTATTTTGGTAGGTGCGCTAG
SEQ ID NO 2:
Nucleotide sequence of AsCqAI gene:
>AsCqAI(bp)
TTGCGAGGAAACCTTCCACTGACCATCGCATTAATGCAGCAGCGAGGAATGCAA
ATCAACCCCCAACTTGTTGATATCATTCTTGGTGAGCGCACCAATGCTCAAGTAA
ATTGTATTCCTAAGTATAACAGGTGTGGGCCTGCTGTGGATGGGGTTCAGTGTTG
CCCTCCTTACACTTGTACCTCACAATACTATGGTAGTTGCTCTTAA
SEQ ID NO 3:
Amino acid sequence of CqBvAI protein:
>CqBvAI(aa)
IRGKVPLLLALRQRGIPVTQSEEAKKIEQELAKIMLEAVAESEYDNNNNNNSALRCIP
QYNRCGPAMDGVPCCAPYTCTSDYFGRCA
SEQ ID NO 4:
Amino acid sequence of AsCqAI protein:
>AsCqAI(aa)
LRGNLPLTIALMQQRGMQINPQLVDIILGERTNAQVNCIPKYNRCGPAVDGVQCCPP
YTCTSQYYGSCS
SEQ ID NO 5:
Amino acid sequence of AhAI_N6M protein:
>AhAI_N6M(32 aa)
CIPKWMRCGPKMDGVPCCEPYTCTSDYYGNCS
SEQ ID NO 6:
Amino acid sequence of AhAI_N6A protein:
>AhAI_N6A(32 aa)
CIPKWARCGPKMDGVPCCEPYTCTSDYYGNCS
SEQ ID NO 7:
Nucleotide sequence of AhAI_N6M gene:
TGTATTCCTAAGTGGATGAGGTGTGGCCCTAAAATGGATGGAGTCCCTTGTTGTG
AACCATACACTTGCACTTCAGACTACTATGGAAATTGCTCTTAA
SEQ ID NO 8:
Nucleotide sequence of AhAI_N6A gene:
TGTATTCCTAAGTGGGCAAGGTGTGGCCCTAAAATGGATGGAGTCCCTTGTTGTG
AACCATACACTTGCACTTCAGACTACTATGGAAATTGCTCTTAA

### Examples

Biological materials used: Amaranthus hypochondriacus leaves were collected from A. hypochondriacus plants naturally occurring in CSIR-NCL campus, Pashan Road, Pune, 411008, India. Alternanthera sessilis leaves were collected from A. sessilis plants which naturally grow in the rainy season in CSIR-NCL campus, Pashan Road, Pune, 411008, India. Beta vulgaris leaves were collected from a vegetable market in Pashan, Pune, 411008, India. Chenopodium quinoa seeds were ordered from Amazon, plants were grown in a greenhouse at CSIR-NCL campus, Pashan Road, Pune, 411008, India. The Callosobruchus chinensis insects were shared by Shivaji University, Kolhapur, 416004, India. Mung beans were purchased from a local market of Pune, 411008, India. It is noted that no microbial strain is covered in the present disclosure; hence, no deposition of strain is done.

### Materials and Methods

RNA isolation, cDNA preparation, and PCR to clone AAIs: Amino acid sequences of AAIs (NCBI ID: XP_021744610.1, accession number: MT627440) and predicted AAI from Beta vulgaris (BvAI) (GenBank accession no. XP_010672947.1) are retrieved from NCBI. Another A. hypochondriacus inhibitor sequence (primary transcript name: AHYPO_000022-RA) is retrieved from available whole-genome data. Leaf tissues of Amaranthus hypochondriacus, Alternanthera sessilis, Chenopodium quinoa, and Beta vulgaris are used to isolate RNA using Spectrum Total Plant RNA Kit (Sigma Aldrich, St. Louis, MO, USA). cDNA is made using M-MLV Reverse Transcriptase (Promega, Madison, WI, USA). Multiple sequence alignment and phylogenetic analysis of AAIsbyClustal omega tool.

Cloning, expression, and purification of recombinant AAIs: ORFs of precursor α-AIs from Amaranthus hypochondriacs (AhAI2_P), Chenopodium quinoa (CqAI_P), and Alternanthera sessilis (AsAI_P); mature peptides of BvAI (BvAI_M), and Amaranthus hypochondriacus (AhAI_M); propeptides of CqAI (CqAI_PP) and AhAI (AhAI_PP) are amplified by designed primer pairs (Table 1). To clone chimaera of pro- and mature peptides, chimeric constructs AsCqAI and CqBvAI are made by overlap extension PCR using overhang primers (Table 1). Amplicons of CqAI_M, BvAI_M, AsAI_PP and CqAI_PP are obtained for further annealing and PCR reactions. The amplified products (AsAI_PP with CqAI_M and CqAI_PP with BvAI_M) are mixed, annealed and extended by five PCR cycles. Moreover, AsCqAI and CqBvAI are amplified using forward primer of AsAI, CqAI, and a reverse primer of CqAI, BvAI, respectively. Amplicons are cloned in the pET28a vector (Novagen, Madison, WI, USA), consisting of N-terminal 6×His-tag. All recombinant plasmids are amplified and sequence characterized.

Recombinant plasmids and empty pET28a (vector control) are further transformed into SHuffle T7 cells of Escherichia coli (New England Biolabs, Ipswich, MA, USA) for protein expression (proteins produced by precursor constructs can be referred to as precursor proteins). When the optical density of cells at 600 nm and at 30° C. reached 0.6, cultures are chilled and induced with 1 mM Isopropyl-β-d-1-thiogalactopyranoside (IPTG). The induced culture is incubated at 16° C., 180 rpm for 14 h. Cell pellets are obtained by centrifugation, followed by the resuspension in lysis buffer (20 mMTris, 300 mMNaCl, and 5% glycerol; pH 7). For cell lysis, the resuspended pellet is subjected to a sonication cycle of 8 sec on 12 sec off cycles at 45% amplitude for 10 mi on ice. Cell debris is separated by centrifugation. The clear cell lysate is kept for binding to previously equilibrated Ni-NTA resin for 1 h at 4° C. This protein-bound slurry is then loaded on Bio Rad gravity column. The column is washed with an increasing concentration of imidazole (10 mM, 25 mM, and 50 mM) in the lysis buffer. Protein is eluted with a buffer containing 250 mM imidazole. Bradford method is used for protein quantification with BSA as standard. Proteins are visualized on 15% SDS-PAGE gel and confirmed by Western blotting.

TABLE 1
Details of primers for cloning of inhibitor gene

| | | | Length |
|---|---|---|---|
| Primer Name | SEQ. ID | Primer sequence | (bp) |
| BvAI_NdeI_pET28aF | SEQ ID NO: 9 | AAAACATATGCGCGAAAATGTTCCTTTGGCC | 31 |
| BvAI_XhoI_pET28aR | SEQ ID NO: 10 | CATTCTCGAGCTACGCGCACCTACCAAAATAG | 32 |
| PrematCqAI_NdeI_F | SEQ ID NO: 11 | GGAATCATATGATCCGGGGAAAAGTC | 26 |
| CqAI_XhoI_R | SEQ ID NO: 12 | GGAATCTCGAGTTAAGAGCAACTACC | 26 |
| PrematAsAI_NdeI_F | SEQ ID NO: 13 | GGGATCATATGTTGCGAGGAAACCTTC | 27 |
| AsAI_XhoI_R | SEQ ID NO: 14 | GGAATCTCGAGTTAAGAGCAATTTCCGTAG | 30 |
| AhAI2_NdeI_pET28aF | SEQ ID NO: 15 | AAAACATATGACCGGGCGACAGAAAATGTGG | 31 |
| AhAI2_XhoI_pET28aR | SEQ ID NO: 16 | CATTCTCGAGTTATATGAAGCTAAATTCATGTATG | 35 |
| CqAIProP_XhoI_R | SEQ ID NO: 17 | GGAATCTCGAGACGTAGAGCGTTG | 24 |
| CqPP_BvMP_F | SEQ ID NO: 18 | ACGCTCTACGTTGTATTCCACAATATAAC | 29 |
| CqPP_BvMP_R | SEQ ID NO: 19 | GTTATATTGTGGAATACAACGTAGAGCGT | 29 |
| AsPP_CqMP_F | SEQ ID NO: 20 | CCAATGCTCAAGTAAATTGTATTCCTAAGTATAAC | 35 |
| AsPP_CqMP_R | SEQ ID NO: 21 | GTTATACTTAGGAATACAATTTACTTGAGCATTGG | 35 |
| MatAhAI_NdeI_F | SEQ ID NO: 22 | GGGGTCATATGTGTATTCCTAAGTGG | 26 |
| AhAI_XhoI_R | SEQ ID NO: 23 | GGAATCTCGAGTTAAGAGCAATTTCCG | 27 |
| AhAI_N6M_F | SEQ ID NO: 24 | GTATTCCTAAGTGGATGAGGTGTGGCCCTAAAAT | 40 |
| | | GGATGG | |
| AhAI_N6M_R | SEQ ID NO: 25 | CCATCCATTTTAGGGCCACACCTCATCCACTTAGG | 40 |
| | | AATAC | |
| AhAI_N6A_F | SEQ ID NO: 26 | GGCAGCCATATGTGTATTCCTAAGTGGGCAAGGT | 39 |
| | | GTGGC | |
| AhAI_N6A_R | SEQ ID NO: 27 | GCCACACCTTGCCCACTTAGGAATACACATATGG | 39 |
| | | CTGCC | |

Determination of molecular weight by MALDI: For MALDI analysis, recombinant proteins of AsAI_P, CqAI_P and CqBvAI (1 mg/ml) are mixed with 10 mg sinapinic acid (Sigma-Aldrich, St Louis, CA, USA) per ml of 50% ACN and 0.1% TFA in a 1:5 ratio. The dry droplet method is used to spot the protein-matrix mixture on the MALDI target plate. AB SciexMALDI-TOF/TOFTM 5800 system (Framingham, MA, USA) is used for analysis with positive linear mode from 2 to 20 kDa.

Inhibitory assays and kinetics of recombinant α-AIs: AAIs' specificity towards coleopteran amylases is analysed. Hence, inventors performed inhibitory activity of recombinant inhibitors against recombinant Tribolium castaneum α-amylase (TcAmy). All inhibitory assays showing insecticidal activity are carried out using DNSA assay as described earlier. Further, various other amylases viz, recombinant Callosobruchus chinensis α-amylase (CcAmy), Helicoverpa armigera α-amylase (HaAmy); commercially available amylases from porcine and human pancreas (PPA and HPA), human saliva (HSA), Aspergillus oryzae (AoAmy), Bacillus licheniformis (BlAmy), diastase and β-amylase from Hordeum vulgare (HvAmy) (Sigma Aldrich, St. Louis, MO, USA) are subjected to inhibitory assays to check specificity profile of newly identified AAIs.

Ex vivo Callosobruchus chinensis adult amylase inhibition: Insects are grown on mung beans under controlled conditions. Adults are collected and crushed in liquid nitrogen to obtain fine tissue powder. Tissue powder is dissolved in a 20 mM citrate phosphate buffer (pH 7,1:6 ratio) and incubated overnight at 4° C. After centrifugation, the supernatant is used as a crude CcAmy source. The inhibition assays are carried out with mature, precursor and chimeric α-AIs using concentrations inhibiting 100% rCcAmy.

Inhibitory potentials and kinetics of recombinant α-AIs: The IC₅₀ of new recombinant AAIs is determined using various inhibitor concentrations at fixed enzyme (TcAmy) and substrate concentrations. The IC₅₀ values of mature, precursor and chimeric α-AIs are compared. Ki of precursor AAIs is determined by keeping fixed enzyme and inhibitor concentrations with varying substrate concentrations in each reaction. The same reactions are carried out with different AI concentrations. All the assays are carried out using independent biological replicates.

Molecular modeling of α-AIs and protein-protein docking with target amylases: The library of protein chimeras is made by exchanging pro-peptides of AhAI, AhAI2, AsAI, CqAI, and BvAI with mature peptides of AhAI2, CqAI, BvAI, and AsAI in possible combinations. Three-dimensional structural models of precursor and chimeric AAIs are predicted using the I-TASSER server. The protein sequences of TcAmy and CcAmy are retrieved from the NCBI database (GenBank accession: NM_001114376.1 and KU641476, respectively). SWISS-MODEL is used to model TcAmy, CcAmy{using TMA crystal structure (PDB ID: 1CLV, chain A) as a template} and mature AAIs{using AhAI (PDB ID: 1CLV, chain I) as a template}. The inhibitor binding with target amylases is predicted using the Z-dock server. The energy of docked complexes is minimized using UCSF chimera 1.10.2 software. These docked complexes are visualized using the PyMol molecular visualization system (pymol.org; DeLano Scientific LLC, USA). The interaction energy is calculated using the PDBe-PISA server.

Saturation mutagenesis and interaction analysis of AhAI and mutants: The amino acid sequence of AhAI is retrieved from the NCBI database (GenBank ID: KU641477). A 32 amino acid mature peptide is selected. Each of the 32 amino acids is individually mutated by replacing them with the remaining 19 amino acids by the DUET server. Mutants with positive free energy (ddG) ≥0.5 Kcal/mol are selected for further analysis. Crystal structures of TMA (PDB ID: 1CLV, Chain A, TMA) and HPA (PDB Id: 1HNY) are retrieved from Protein Data Bank (https://www.rcsb.org/). The Duet server predicted the mutant structure. Selected mutants and wild-type AhAI (PDB ID: 1CLV, Chain I) are docked with TMA, TcAmy and HPA by the Z-DOCK server. Docking is performed by selecting the catalytic residues of α-amylases and corresponding inhibitor binding residues from the AhAI_TMA complex (PDB Id: 1CLV). Energy minimization and calculation are done. The energy of H-bonds (1H-bond≈0.5 Kcal/mol) and salt bridges (1 salt bridge≈0.3 Kcal/mol) added to energy calculated by PDBe-PISA. The mutant complexes with higher free energy than wild-type are selected for further analysis. The mutant complex with the highest dG is also analyzed by docking the same mutant with CcAmy. Differences in interactions of wild-type and mutant complexes are studied in silico using PyMol visualization software. Six mutants having higher affinity to TcAmy are selected. These mutants' possible combinations are made in AhAI to create double and triple mutants and then docked with TcAmy to check interaction energies.

Molecular dynamics simulation of TcAmy-AAI complexes: The energy-minimized docked complexes of different amylase inhibitors with TcAmyare used for MD simulation studies (chimera, precursor, mutant and mature inhibitors in complex with TcAmy). The input files for MD simulation are generated using the CHARMM-GUI server with the Monte-Carlo method. Further, the energy minimization is performed with the steepest descent algorithm using 50000 steps and is set to stop when the maximum force per nm decreases below 1000 kJ/mol. It is then equilibrated for 1250 ps at 303.15K using Nos6-Hoover thermostat. GROMACS (version 2020.1) performed a 10 ns production run with Nos6-Hoover thermostat, Parrinello-Rahman pressure coupling and default parameters. The RMSD and RMSF plots of the protein complex are created and visualized using XMGRACE (version 5.1.25). These protein complexes are uploaded to PDBePISA server to study interaction between proteins in complex.

Site-directed mutagenesis and experimental analysis of AhAI and mutants: Forward and reverse mutagenic primers containing the desired mutation are designed (Table 1). Site-directed mutagenesis of AhAl is performed using Quikchange Lightning site-directed mutagenesis kit as per manufacturer protocol (Table 4) (Agilent Technologies, Santa Clara, USA). The amplified products are digested by DpnI at 37° C. for 5 min to digest the parental (i.e., non-mutated) supercoiled double-stranded DNA. The mutated plasmids are transformed into E. coli TOP 10 competent cells. Colony PCR and sequence characterization are performed to check the insert with the mutation's presence. The recombinant plasmids (wild-type, empty vector and mutants N6M, N6A) are transformed and proteins are expressed as mentioned for precursor proteins. Further, cell pellets are resuspended in a buffer (20 mMTris, 300 mMNaCl, 0.5% Triton X-100 pH 8.7). The cell lysate is prepared by centrifugation after sonication. The recombinant protein is purified from the cell lysate using the Ni-NTA chromatography protocol that is previously described. Most bacterial proteins have Pi between 4 to 7; the bacterial protein contaminants are precipitated by changing the pH to 4 by diluted HCl. At pH 4, the samples are gently agitated for 120 min in the cold. The precipitated proteins are then removed by centrifugation at 12000 rpm, 4° C. for 30 min. Again, the pH is neutralized by NaOH. Protein from the supernatant is loaded on 18% Tricine-SDS PAGE and checked for inhibitory activity against TcAmy. α-Amylase inhibition assays and IC₅₀ studies are carried out as previously described for other recombinant inhibitors. A comparison of IC₅₀ values for wild-type and mutant proteins is made (crude and purified). rTcAmy, rCcAmy, HPA (Sigma Aldrich, MO, USA), and HSA (Sigma Aldrich, MO, USA) are subjected to inhibitory assays by these inhibitors.

### Results

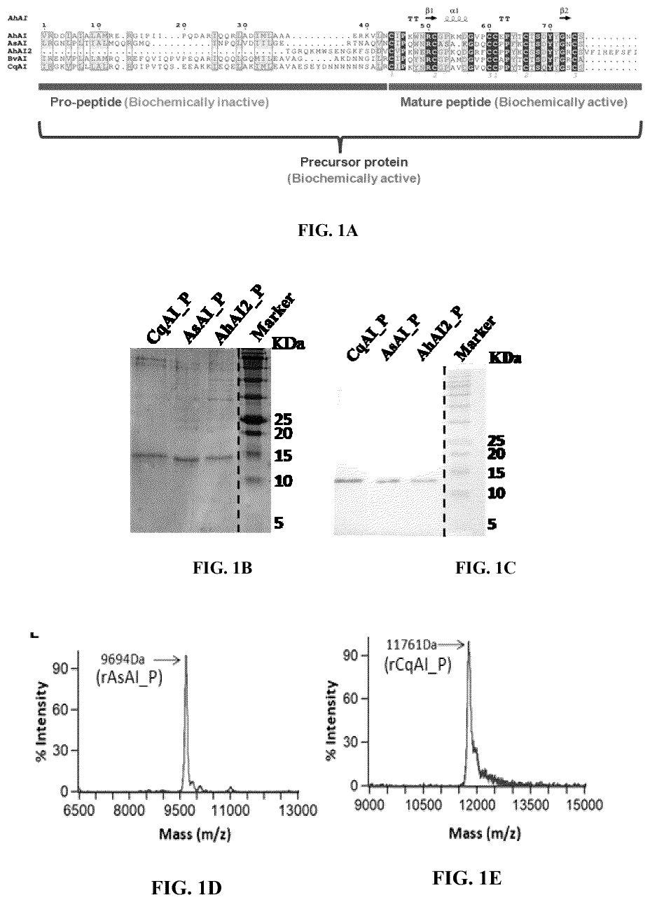

BvAI is a competitive and specific inhibitor of coleopteran amylases: The putative α-AI, BvAI_M shares similar secondary structural characteristics and disulfide bond topology with other AAIs (FIG. 1A). It competitively and specifically inhibits coleopteran amylases with an IC₅₀ value of 41.06 μM and Ki value of 19.31 μM.

Precursor AAIs possess an α-amylase inhibitory potential: AAI genes are composed of pro and mature peptides; mature peptides mainly contribute to the inhibitory activity. Likewise, AI extracted as an active protein from Amaranthus hypochondriacus tissue consists of only mature peptide parts. Due to lack of evidence, the role of propeptide in the generation of mature AAI is still enigmatic. The length of AAI pro-peptides varies from 17 to 55 amino acids (FIG. 1A). As pro-peptide sequences are highly diverse, it is more difficult to predict or annotate their function in AAI structure or activity. To elucidate pro-peptide functionality, inventors cloned and purified precursor proteins (FIG. 1B). Expression of these proteins is confirmed by Western blotting (FIG. 1C). Molecular weight of AsAI_P and CqAI_P proteins is 9694 Da (m/z) and 11761 Da (m/z), respectively. (FIGS. 1D-IE) as determined by MALDI-TOF analysis which correlates with their theoretical molecular mass 9853 Da and 11944 Da respectively.

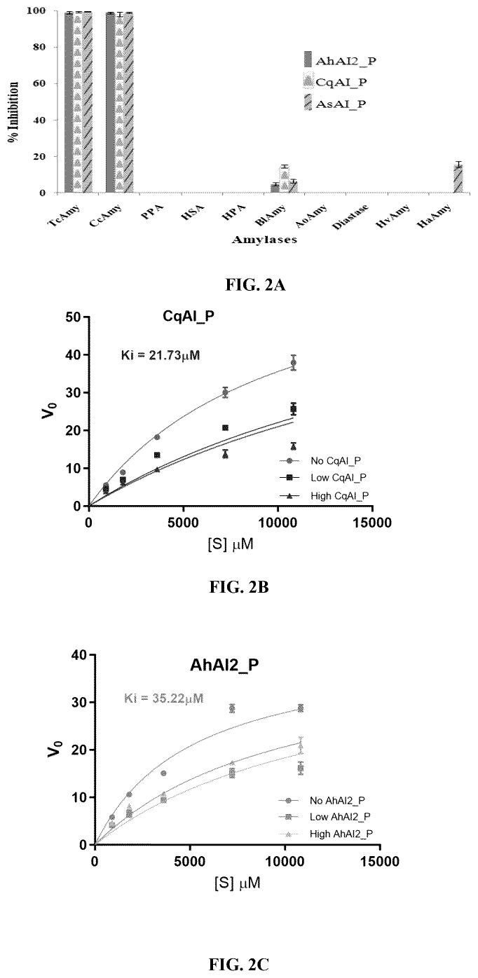

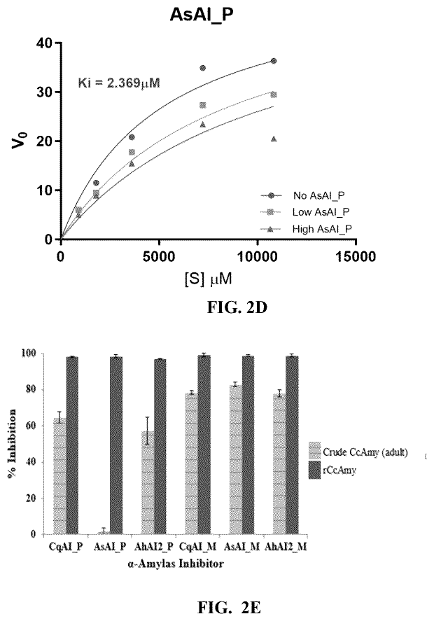

Differential interactions of mature and precursor α-AIs with TcAmy: It has been observed that IC₅₀ values for mature and precursor α-AIs differ (Table 2). IC₅₀ of proteins AhAI2 mature (AhAI2_M) and AhAI2 precursor (AhAI2_P), is found to be ˜22 μM and ˜67 μM respectively. Conversely, CqAI_P and AsAI_P have IC₅₀ values of 57.60 μM and 6.85 μM, respectively, which are much less than the IC₅₀ of CqAI_M and AsAI_M (103.1 μM and 89.14 μM) (Table 2). Furthermore, precursor α-AIs retained the specificity toward Coleopteran amylases (TcAmy and CcAmy) like mature proteins (FIG. 2A) and negligible or no inhibition of other amylases (FIG. 2A). Ki values of AhAI2_P, AsAI_P, and CqAI_P are 35.22 μM, 2.37 μM, and 21.73 μM, respectively (FIGS. 2B-2D) which showed a competitive mode of inhibition. Similar to their mature counterpart, these precursor AAIs are found to be active against crude CcAmy (FIG. 2E). Inhibition potential of AAIs with crude and recombinant CcAmy is found to be different. (FIG. 2E). This difference may be due to various ex vivo factors in crude CcAmy such as multiple isoforms.

TABLE 2
Differences in IC₅₀ values of mature and precursor AAIs

| Sr. No. | α-AI | IC₅₀values (μM) | |
|---|---|---|---|
| 1. | AsAI_M | 89.14 | (±4.3) |
| 2. | AsAI_P | 6.85 | (±0.16) |
| 3. | CqAI_M | 103.1 | (±5.7) |
| 4. | CqAI_P | 57.60 | (±0.55) |
| 5. | AhAI2_M | 22.03 | (±1.68) |
| 6. | AhAI2_P | 67 | (±3.34) |

(*M denotes mature peptide/protein, and P denotes precursor peptide/protein. Also, the mature peptide is a natural peptide obtained as it is from the microorganism as listed above)

Recombinant precursor proteins, AsAI_P, CqAI_P, and AhAI2_P possess α-amylase inhibitory activity, despite highly variable propeptides. Furthermore, to assess the inhibitory activity of pro-peptides, propeptides of AhAI and CqAI are cloned and expressed. These recombinant proteins do not show inhibitory activity against TcAmy. This indicates that propeptide alone does not have intrinsic amylase inhibitory activity. Still, it may play a crucial role in regulating structure-function of mature AAIs. The molecular mechanism behind differential inhibition caused by mature and precursor AAIs is elucidated by in silico analysis. Predicted models of precursor α-AIs showed that the pro and mature peptides form two distinct domains. Precursor α-AIs complexed with TcAmy showed tight binding and packing of mature peptides in the catalytic groove of α-amylase. Binding of mature peptide in the active site results in inaccessibility of the substrate, thus inhibiting its activity. In a few cases, it has been observed that propeptide also interacts at the interface of the enzyme-inhibitor complex, assisting the complex interaction. This difference in interaction and interacting residues of mature and precursor α-AIs with TcAmy may cause the difference in their IC₅₀ values. Precursor AAI establishes multiple ancillary contacts with TcAmy and may strengthen mature peptide interaction (Table 3).

TABLE 3
Differences in the interactions of mature and precursor AAIs with TcAmy

| | Complex | | | | Interface | N_{HB} | | |
|---|---|---|---|---|---|---|---|---|
| Sr. | with | Interacting residues at catalytic site | | ΔG | Area | Catalytic | Interface | |
| No. | TcAmy | TcAmy | α-AI | (Kcal/mol) | (A²) | site | (Total) | N_{SB} |
| 1 | AhAI2_M | Asp-187, Glu-224, | Arg-7, Arg-30 | −9.6 | 1185.5 | 6 | 11 | 10 |
| | | Asp-289 | | | | | | |
| 2 | AhAI2_P | Asp-289 | Lys-21, Trp-22, | −9.9 | 1230.2 | 3 | 7 | 0 |
| | | | Glu-23 | | | | | |
| 3 | CqAI_M | Asp-187, Glu-224, | Arg-7, Tyr-28, | −14.8 | 1101.5 | 5 | 10 | 4 |
| | | Asp-289 | Ser-30 | | | | | |
| 4 | CqAI_P | Glu-224, Asp-289 | Asn-61, Arg-62 | −8.6 | 1347.8 | 2 | 11 | 4 |
| 5 | AsAI_M | Asp-187, Glu-224, | Arg-7, Tyr-27, | −10.6 | 1111.6 | 6 | 15 | 4 |
| | | Asp-289 | Asn-29 | | | | | |
| 6 | AsAI_P | Asp-187, Asp-289 | Lys-58, Thr-60, | −15.7 | 1227.1 | 3 | 7 | 1 |
| | | | Asn-66 | | | | | |

Chimeric AAIs showed enhanced inhibitory potential without compromising specificity: The difference in inhibition potential and molecular interaction of mature and precursor AAIs with TcAmy hinted at generating the chimera library with multiple combinations of mature and propeptide regions. Chimera inhibitor peptide constructs are developed to understand (i) the importance of propeptide in differential inhibition and (ii) the possibility of enhancing the inhibitory potential of natural proteins by exchanging propeptides of mature AAIs. Predicted models of Chimera inhibitor peptide constructs showed that a change in any of two domains changes the conformation of adjacent domain.

Furthermore, to understand the effect of propeptide in chimera structure and its impact on the interaction with target amylase, inventors performed in silico study by creating the combination of mature peptide (CqAI) with varying propeptides. The interaction analysis showed that along with the change in AI conformation, the total interface area and interacting residues also changed (Table 4). Interaction differences are majorly contributed by variation in the propeptide region of α-AIs with its contacts with TcAmy. Propeptides of AsCqAI and Ah1CqAI do not interact with TcAmy. In rest complexes, both pro- and mature peptides interact with TcAmy. Based on enhanced binding potential, the chimera inhibitor peptide construct CqBvAI (CqAI_PP and BvAI_M) is selected for further experimental analysis. Inhibition kinetics showed that the inhibition potential of AsAI_M increased as IC₅₀ decreased significantly, when it is attached to AsAIpropeptide (AsAI_P). Moreover, it has been found that IC₅₀ value of CqAI_M is higher than others; hence AsCqAI chimera (AsAI_PP and CqAI_M) is created to assess AsAI_PP effect on the inhibitory potential of CqAI_M.

TABLE 4
In silico interaction details of precursor and chimera inhibitor peptides of CqAI with TcAmy

| | Complex | Interacting residues | | Interacting residues | | | | | |
|---|---|---|---|---|---|---|---|---|---|
| Sr. | with | | α-AI | | α-AI | ΔG | Interface | N_{HB} at | |
| No. | TcAmy | TcAmy | (MP) | TcAmy | (PP) | (Kcal/mol) | Area (A²) | interface | N_{SB} |
| 1. | CqAI_P | Glu-224, | Asn-61, | Asn-55, | Arg-14, | −8.6 | 1347.8 | 11 | 4 |
| | | Asp-289, | Arg-62, | Asn-333, | Gln-13, | | | | |
| | | Glu-231, | Lys-59, | Ser-296, | Val-39, | | | | |
| | | Val-138, | Thr-79 | Gln-297 | Asn-51, | | | | |
| | | Val-153 | | | Arg-55 | | | | |
| 2. | Ah1CqAI | Glu-231, | Tyr-64, | — | — | −11.9 | 1213.2 | 11 | 0 |
| | | Ile-226, | Lys-47, | | | | | | |
| | | Asp-289, | Asn-49, | | | | | | |
| | | Val-138, | Thr-67, | | | | | | |
| | | | Ser-73, | | | | | | |
| 3. | Ah2CqAI | Gly-294, | Ser-49, | Tyr-141, | Asp-15, | −11.0 | 1204.8 | 13 | 2 |
| | | Asp-289, | Ser-47, | Asn-139 | Asp-16 | | | | |
| | | Asp-187 | Arg-24 | | | | | | |
| 4. | BvCqAI | Glu-224, | Tyr-79, | Ser-53, | Gln-25, | −9.5 | 1198.1 | 11 | 4 |
| | | Asp-334, | Arg-58 | Asn-55, | Arg-27, | | | | |
| | | Asn-333 | | Asn-332 | Gln-30 | | | | |
| 5. | AsCqAI | Gln-63, | Tyr-64, | — | — | −12.6 | 1208.4 | 4 | 0 |
| | | Asn-333, | Gln-53, | | | | | | |
| | | Lys-190, | Ser-67, | | | | | | |
| | | Asp-289 | Ser-62 | | | | | | |
| 6. | AsAI_P | Asp-187, | Lys-58, | — | — | −15.7 | 1227.1 | 7 | 1 |
| | | Asp-289, | Asn-66, | | | | | | |
| | | Ile-298, | Tyr-64, | | | | | | |
| | | Gln-297 | Tyr-63 | | | | | | |
| 7. | BvAI_P | Asp-289, | Arg-81, | Thr-293 | Gln-25 | −6.7 | 1021 | 10 | 3 |
| | | Lys-190, | Ser-76, | | | | | | |
| | | Asn-139 | Thr-73 | | | | | | |
| 8. | CqBvAI | Glu-231, | Arg-62, | Thr-137 | Gln-13 | −15.9 | 1247.3 | 12 | 6 |
| | | Asp-289 | Gly-84, | | | | | | |
| | | | Tyr-82 | | | | | | |

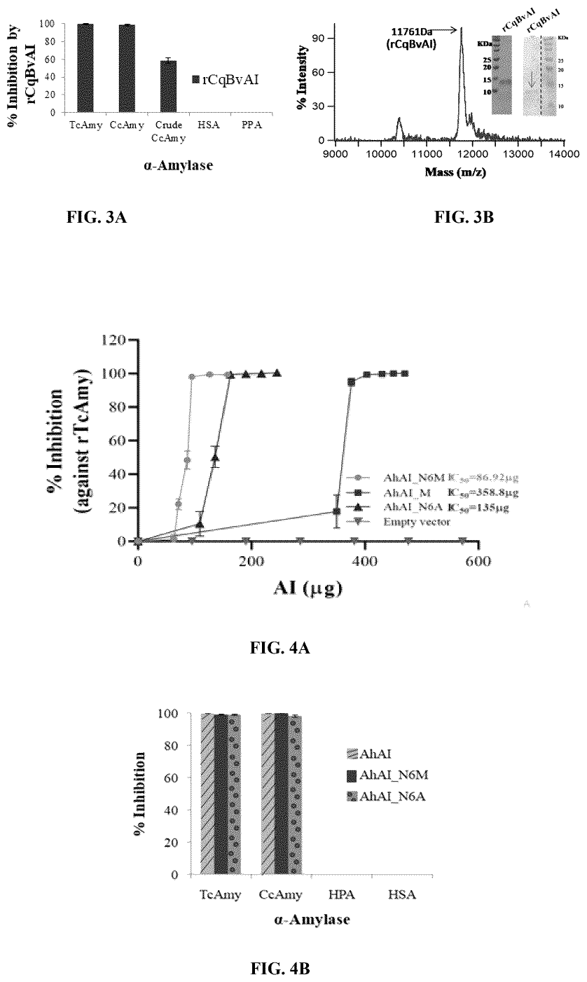

Experimental analysis of chimera inhibitor peptide constructs showed that they retained the inhibitory activity and specificity. The accurate molecular weight of CqBvAI is 11761 Da (m/z) as found by MALDI-TOF analysis and 11943 Da by theoretical calculation (FIG. 3A). Inventors observed a variation in IC₅₀ values of chimera inhibitor peptides and wild-type proteins. When CqAI_PP is attached to BvAI_M, forming CqBvAI, the IC₅₀ value is reduced from 41.06 μM to 13.51 μM (Table 5). The CqBvAI is more efficient than the CqAI_P, CqAI_M and BvAI_M (Table 5). Hence, replacing CqAI_PP before BvAI_M increased the potency of BvAI. CqBvAI is 7.6 and 4.26 times more potent than CqAI_M and CqAI_P, respectively. In silico interaction analysis showed that though CqAI_P forms more H-bonds than CqBvAI, dG is more negative for CqBvAI than CqAI_P, resulting in more vital interaction with TcAmy. The protein produced by AsCqAI is less potent for TcAmy inhibition than AsAI_P (Table 5), which correlated with experimental and in silico data (Table 5). The in silico interaction studies showed that AsAI_P has more affinity to TcAmy and forms more H-bonds than AsCqAI (Table 4). The experimental analysis supports this data as the IC₅₀ value of AsCqAI (15.91 μM) is more than AsAI_P (6.89 μM) (Table 5). Compared to CqAI_M, for AsCqAI, the IC₅₀ value is reduced to 6.48 times, increasing inhibition potential astoundingly. AsCqAI is more potent than CqAI_P, AsAI_M, and CqAI_M proteins (Table 5). The chimeric α-AIs retained the specificity toward coleopteran amylases (FIG. 3B).

TABLE 5
Comparison of IC₅₀ values of chimera inhibitor
peptides with mature and precursor proteins

| α-AI | IC₅₀ (μM) | |
|---|---|---|
| CqAI_M | 103.1 | (±5.7) |
| CqAI_P | 57.60 | (±0.5) |
| BvAI_M | 41.06 | (±0.43) |
| CqBvAI | 13.53 | (±3.3) |
| AsAI_M | 89.14 | (±4.3) |
| AsAI_P | 6.89 | (±0.17) |
| AsCqAI | 15.91 | (±0.13) |

(Here, M denotes mature peptide/protein, and P denotes precursor peptide/protein. Also, the mature peptide is a natural peptide obtained as it is from the microorganism as listed above; Also CqBvAI and AsCqAI is a mutated peptide prepared in-vitro)

The molecular dynamic simulation of TcAmy-AI complexes showed that the interactions remain stable. Few complexes showed initial fluctuation, but after 5 ns, they became stable. The affinity of chimeric construct CqBvAI (ΔⁱG=−13.8 Kcal/mol) is higher towards TcAmy than its parental counterparts except for AsAI_P. These energy results correspond to experimental results as the IC₅₀ value of CqBvAI (13.53 μM) is less than the rest AIs except AsAI_P (Table 5). However, said natural peptide AsAI_P may show resistance by the continuously evolving nature of the insects. In contrast to this, the claimed mutated peptide (alone) may not show resistance. In other words, CqBvAI and AsCqAI are mutated peptides with inhibitory activity better than said mature peptides and natural peptides. Even if any natural peptide shows better inhibition than the claimed CqBvAI and AsCqAI peptides in Table 5, said natural peptide will be more prone of resistance by the continuously evolving nature of the insects as compared to the mutated peptide (alone) as disclosed herein this disclosure.

Saturation mutagenesis generated specific and hyperactive mutants: Along with chimera formation, site saturation mutagenesis further improved AAI inhibition potential. Saturation mutagenesis of AhAI by the Duet server showed many stable mutations (171 out of 608). The mutants having solvation-free energy ≥0.5 are selected for further analysis. Complexes of selected mutants and wild-type inhibitors with TcAmy showed that six mutant-TcAmy complexes have solvation-free energy greater than wild-type-TcAmy complexes. These included N6L, N6M, K11I, N30L, N30M, N30V mutations (Table 6). From these, N6 belongs to segment-1 (part of β-turn) of AhAI, which is involved in amylase inhibitory activity. N30 belongs to segment-5 (part of the β-strand) and is also a part of the reactive site loop of AhAI. In contrast, K11 belongs to segment-2 (part of β-turn), which is involved in inhibitor folding. The mutant analysis highlighted the importance of S1 in inhibition and S2 in folding.

Further, the interaction of selected mutants is checked by docking them with TMA and HPA. Inventors found 11 and 9 docked complexes with solvation-free energy greater than wild-type complexes, respectively. Six mutants that showed tighter binding with these amylases are selected. Different combinations of these six mutants projected double and triple mutants. Their docking with TcAmy showed that N6M has the highest affinity than any double and triple mutant. N6M showed the highest affinity for binding (lowest AG) when complexed with TcAmy, CcAmy and TMA. Docking of this mutant with CcAmy, showed a decrease in AG value from −15.6 Kcal/mol to −18 Kcal/mol. Hence, mutant AhAI_N6M has been selected for experimental analysis along with AhAI_N6A. N6M mutation is a replacement of polar residue by non-polar. The superimposition of TcAmy-AhAI and TcAmy-AhAI_N6M complexes showed that the Met with a long linear side chain penetrates more inward in the active site groove than Asn with a Y-shaped side chain. The replacement of polar residue Asn by non-polar Met showed that the H-bonds formed by Asn-6 with Glu-231 and Lys-190 are lost. M does not create H-bonds with any amylase residue. Due to changes in shape, length, and polarity of amino acids, position of inhibitor residues changes inside the active site groove resulting in changes in interacting residues. Due to this, there is an increase in the number of H-bonds formed by a mutant at the interface of enzyme-inhibitor complex than AhAI-TcAmy along with an increase in interface area and AG. The interaction energy calculated after molecular dynamic simulation also showed a stronger affinity of AhAI_N6M towards TcAmy than wild-type AhAI. Inventors also observed an increase in interface area along with the number of the H-bonds and salt bridges formed by mutant AhAI_N6M than wild type AhAI. The root-mean-square-deviation (RMSD) plot showed stable interaction. The root-mean-square-fluctuation (RMSF) analysis of wild-type and mutant protein complexes indicated that the interacting residues in the complex remained stable over the simulation with fluctuations at few residues. These fluctuations may be why the mutant is a more potent inhibitor of TcAmy than the wild-type inhibitor, which also correlates with the experimental data.

TABLE 6
Energies of interaction higher than wild-type
by mutants when docked with TcAmy, TMA and HPA

| | AhAI | ΔG (Kcal/mol) | | | Location in |
|---|---|---|---|---|---|
| Sr. No. | Mutant | TcAmy | TMA | HPA | the inhibitor |
| | Wild-type | −14.6 | −14.2 | −12.8 | |
| 1. | N6L | −15.3 | −16.3 | −12.8 | Segment 1 |
| 2. | N6M | −18.5 | −18.2 | −13.6 | |
| 3. | K11I | −15.7 | — | −13.8 | Segment 2 |
| 4. | N30L | −14.9 | −16.1 | −14.9 | Segment 5 |
| 5. | N30M | −15.5 | — | −15.9 | |
| 6. | N30V | −15.3 | −15.2 | −14.6 | |

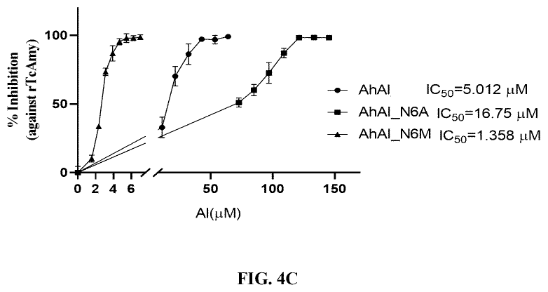

Inhibitory assays with crude samples of AhAI and its mutants showed that both are active against TcAmy (FIG. 4A-C). AhAI_N6M is four times more potent than wild-type AhAI. IC50 values of AhAI_M, AhAI_N6M, and AhAI_N6A are 358.8 μg, 86.92 μg, and 135 μg, respectively. Inhibitory assays with purified AhAI and its mutants showed that AhAI_N6M is approximately 3.4 times more potent than wild-type AhAI (FIG. 4A). Empty vector protein showed no inhibition of TcAmy (FIG. 4B). The affinity of AhAI_N6M is more for TcAmy, TMA, and HPA. The interaction energy is more negative for AhAI_N6M with TcAmy, CcAmy, and HPA. Inventors observed that, like AhAI, AhAI_N6M and AhAI_N6A do not inhibit HPA and HSA and ultimately inhibit recombinant TcAmy and CcAmy. Mutants of AhAI are specific to the Coleopteran insect amylases like that of wild-type AhAI (FIG. 4C).

A discrepancy in inhibitions by precursor and mature AAIs is observed due to the formation of differential contacts with TcAmy. In silico interaction studies showed that the combination of pro- and mature peptides changes the conformation of the inhibitor resulting in changes in the interacting residues of α-amylase and α-AI. These changes altered the potency of the inhibition without altering specificity. Also, recombinant α-AIs produced from mature constructs are active. Hence, propeptide may not be essential to produce active recombinant mature inhibitors in vitro. In the case of ω-conotoxins, it is proved that they do not need propeptide for folding. The mature form of a few ω-conotoxins has sufficient information for correct folding. The mutagenic study of AhAI demonstrated that segment 2 of AhAI M is involved in folding as mutations in it affected the folding of mature peptides. However, the inhibition profile of proteins produced by mature and precursor peptides vary greatly. The attachment of propeptides to mature peptides changed the efficiency of protein. This may be because of a change in protein conformation, interaction energies due to a variation in interacting residues, enzyme and inhibitor interface area, number of H-bonds, and salt bridges formed. However, there is a possibility that in plants, the N-terminal propeptide may direct cellular routing or mediate interactions with regulatory proteins. There is also the possibility that, like a few cystine-knot growth factors, they may function in the storage of AIs at specific locations or be involved as stress sensors. Few precursor AAIs proved more effective than their mature counterparts hence domains of AAIs are shuffled to make superior inhibitors. Recombinant proteins produced by chimeric constructs CqBvAI and AsCqAI showed tighter binding to TcAmy and hence more efficient than their parental counterparts except for CqAI_P. They also retained the specificity towards coleopteran amylases. Hence with the help of the domain shuffling approach potency of inhibitors is improved. Another protein engineering approach used is site saturation followed by site-directed mutagenesis, which produces specific inhibitors superior to the natural ones. Inventors observed that AhAI accommodated mutations N6M and N6A retaining activity. This is a replacement of polar residue (N) with a Y-shaped side chain by non-polar residue with a long linear side chain (M) or non-bulky, chemically inert methyl functional group as a side chain (A). This 6th position ‘N’ may play an important role in inhibition as it is conserved in other AAIs except for AhAI2. But its replacement with ‘M’ or ‘A’ produced hyperactive mutants.

At high temperatures, ‘N’ in proteins undergodeamidation, which may result in protein misfolding and inactivation. Therefore, without being bound to a particular theory, it is believed that replacement of ‘N’ may be responsible for increasing the stability of the protein. Hence, these mutants may prove beneficial in making pest control formulations. All the α-AIs produced by protein engineering are hyperactive and specific toward coleopteran amylases. Here, the specificity is achieved for plant-insect interaction; hence, safe for the ecosystem. These inhibitors can be used to control coleopteran pests by making formulations for pest control or generating transgenic crops or plants. The application of combinations of these different inhibitors could be effective in controlling the emergence of resistance. It can prove to be advantageous as an addition to pest control technology. Another added advantage of these inhibitors is non-toxicity hence imparting less evolutionary pressure by retarding growth rather than killing insects. This study has led to the efficient development of eco-friendly pest control technology for stored grains.

The present disclosure provides a modified variant of Amaranthaceae α-amylase inhibitors (hereinafter referred to as AAIs) having stronger affinity towards target amylases. The present disclosure provides peptide and propeptide with 2-to-8-fold higher efficacy or inhibition potential. The present disclosure provides a modified variant of AAI which retains specificity towards target coleopteran α-amylases. The present disclosure provides a modified variant of AAI i.e., recombinant Amaranthus hypochondriacus α-AI with increased specific inhibition potential. The present disclosure provides formulations for pest control comprising a modified variant of AAI which retains specificity towards target coleopteran α-amylases. The present disclosure provides methods for generating transgenic crops or plants comprising nucleic acid molecules that code for modified variants of AAI wherein AAI retains specificity towards target coleopteran α-amylases. The present disclosure provides methods for increasing plant resistance, generating transgenic plants which are capable of expressing genes that encode for molecules having improved amylase inhibiting activity.

1. A chimera amylase inhibitor peptide having an amino acid sequence selected from SEQ ID NOs: 3-6.

2. The chimera inhibitor peptide of claim 1, wherein the chimera amylase inhibitor peptides are Amaranthaceae α-amylase inhibitors.

3. The chimera inhibitor peptide of claim 1, wherein the chimera amylase inhibitor peptide comprises a mutation in one or more mature peptide regions.

4. The chimera inhibitor peptide of claim 3, wherein the mutation is selected from the group consisting of N6L, N6M, K11I, N30L, N30M, and N30V.

5. A polynucleotide sequence encoding the chimera inhibitor peptide of claim 1, wherein the polynucleotide sequence is selected from the group consisting of SEQ ID NOs: 1, 2, 7 and 8.

6. A vector comprising a polynucleotide sequence selected from the group consisting of SEQ ID NOs: 1, 2, 7 and 8.

7. A construct comprising a promoter region operably linked to a nucleotide sequence having 70 to 75% similarity with a nucleotide sequence selected from the group consisting of SEQ ID NOs: 1, 2, 7 and 8.

8. The construct of claim 7, wherein the construct further comprises
a termination signal;
a replication origin;
a selection marker; and
a cloning site.


SEQUENCE LISTING (27 sequences)

SEQ ID NO: 1 - DNA, 263 bp, synthetic construct
  source 1..263: mol_type = other DNA
atccggggaaaagtcccgttgttgctcgccctgcgacaacgaggaatcccggtgacacaatcggaagaagcgaaaaagattgagcaagagcttgcaaaaatcatgctagaagctgttgctgaatctgagtacgacaacaacaacaataacaacagcgctctacgttgtattccacaatataacaggtgtggtcctgcaatggatggagttccttgttgtgctccatacacatgcacctcagactattttggtaggtgcgctag

SEQ ID NO: 2 - DNA, 210 bp, synthetic construct
  source 1..210: mol_type = other DNA
ttgcgaggaaaccttccactgaccatcgcattaatgcagcagcgaggaatgcaaatcaacccccaacttgttgatatcattcttggtgagcgcaccaatgctcaagtaaattgtattcctaagtataacaggtgtgggcctgctgtggatggggttcagtgttgccctccttacacttgtacctcacaatactatggtagttgctcttaa

SEQ ID NO: 3 - protein, 87 aa, synthetic construct
IRGKVPLLLALRQRGIPVTQSEEAKKIEQELAKIMLEAVAESEYDNNNNNNSALRCIPQYNRCGPAMDGVPCCAPYTCTSDYFGRCA

SEQ ID NO: 4 - protein, 69 aa, synthetic construct
LRGNLPLTIALMQQRGMQINPQLVDIILGERTNAQVNCIPKYNRCGPAVDGVQCCPPYTCTSQYYGSCS

SEQ ID NO: 5 - protein, 32 aa, synthetic construct
CIPKWMRCGPKMDGVPCCEPYTCTSDYYGNCS

SEQ ID NO: 6 - protein, 32 aa, synthetic construct
CIPKWARCGPKMDGVPCCEPYTCTSDYYGNCS

SEQ ID NO: 7 - DNA, 99 bp, synthetic construct
  source 1..99: mol_type = other DNA
tgtattcctaagtggatgaggtgtggccctaaaatggatggagtcccttgttgtgaaccatacacttgcacttcagactactatggaaattgctcttaa

SEQ ID NO: 8 - DNA, 99 bp, synthetic construct
  source 1..99: mol_type = other DNA
tgtattcctaagtgggcaaggtgtggccctaaaatggatggagtcccttgttgtgaaccatacacttgcacttcagactactatggaaattgctcttaa

SEQ ID NO: 9 - RNA, 31 nt, synthetic construct
  source 1..31: mol_type = other RNA
aaaacatatgcgcgaaaatgttcctttggcc

SEQ ID NO: 10 - RNA, 32 nt, synthetic construct
  source 1..32: mol_type = other RNA
cattctcgagctacgcgcacctaccaaaatag

SEQ ID NO: 11 - RNA, 26 nt, synthetic construct
  source 1..26: mol_type = other RNA
ggaatcatatgatccggggaaaagtc

SEQ ID NO: 12 - RNA, 26 nt, synthetic construct
  source 1..26: mol_type = other RNA
ggaatctcgagttaagagcaactacc

SEQ ID NO: 13 - RNA, 27 nt, synthetic construct
  source 1..27: mol_type = other RNA
gggatcatatgttgcgaggaaaccttc

SEQ ID NO: 14 - RNA, 30 nt, synthetic construct
  source 1..30: mol_type = other RNA
ggaatctcgagttaagagcaatttccgtag

SEQ ID NO: 15 - RNA, 31 nt, synthetic construct
  source 1..31: mol_type = other RNA
aaaacatatgaccgggcgacagaaaatgtgg

SEQ ID NO: 16 - RNA, 35 nt, synthetic construct
  source 1..35: mol_type = other RNA
cattctcgagttatatgaagctaaattcatgtatg

SEQ ID NO: 17 - RNA, 24 nt, synthetic construct
  source 1..24: mol_type = other RNA
ggaatctcgagacgtagagcgttg

SEQ ID NO: 18 - RNA, 29 nt, synthetic construct
  source 1..29: mol_type = other RNA
acgctctacgttgtattccacaatataac

SEQ ID NO: 19 - RNA, 29 nt, synthetic construct
  source 1..29: mol_type = other RNA
gttatattgtggaatacaacgtagagcgt

SEQ ID NO: 20 - RNA, 35 nt, synthetic construct
  source 1..35: mol_type = other RNA
ccaatgctcaagtaaattgtattcctaagtataac

SEQ ID NO: 21 - RNA, 35 nt, synthetic construct
  source 1..35: mol_type = other RNA
gttatacttaggaatacaatttacttgagcattgg

SEQ ID NO: 22 - RNA, 26 nt, synthetic construct
  source 1..26: mol_type = other RNA
ggggtcatatgtgtattcctaagtgg

SEQ ID NO: 23 - RNA, 27 nt, synthetic construct
  source 1..27: mol_type = other RNA
ggaatctcgagttaagagcaatttccg

SEQ ID NO: 24 - RNA, 40 nt, synthetic construct
  source 1..40: mol_type = other RNA
gtattcctaagtggatgaggtgtggccctaaaatggatgg

SEQ ID NO: 25 - RNA, 40 nt, synthetic construct
  source 1..40: mol_type = other RNA
ccatccattttagggccacacctcatccacttaggaatac

SEQ ID NO: 26 - RNA, 39 nt, synthetic construct
  source 1..39: mol_type = other RNA
ggcagccatatgtgtattcctaagtgggcaaggtgtggc

SEQ ID NO: 27 - RNA, 39 nt, synthetic construct
  source 1..39: mol_type = other RNA
gccacaccttgcccacttaggaatacacatatggctgcc